(19) United States
(12) Patent Application Publication
(10) Pub. No.: US 2026/0131088 A1
(43) Pub. Date: May 14, 2026

(54) VEIN SAVER

(71) Applicant: Cory SCHIERBERL, Columbus, OH (US)

(72) Inventor: Cory SCHIERBERL, Columbus, OH (US)

(21) Appl. No.: 19/300,046

(22) Filed: Aug. 14, 2025

Related U.S. Application Data
(60) Provisional application No. 63/718,612, filed on Nov. 9, 2024.

(51) Int. Cl. A61M 5/42

(52) U.S. Cl. CPC A61M 5/427; A61M 2205/502

(57) ABSTRACT

Systems and methods are described for tracking venipunctures, calculating venipuncture risk, and displaying said risk for the user. Systems and methods are also disclosed which may enable determining suitable sites for injection of a particular treatment, calculating the local and composite effects from the injection at each suitable injection site, and selecting a recommended suitable injection site for the user.

### CROSS-REFERENCE TO RELATED APPLICATIONS

This patent application claims priority to and the benefit of pending provisional patent application 63/718,612 filed Nov. 9, 2024, which is incorporated herein by reference in its entirety.

### TECHNICAL FIELD

The present disclosure generally relates to maintaining vascular health throughout an individual's lifespan and, more particularly, to utilizing venipuncture historical location data to identify and mitigate potential future injuries for vascular longevity purposes.

### BACKGROUND

Every time a vein is punctured, it can scar. Repeated injections in the same or nearby locations result in progressive scarring of the vein, pain, difficulty when reusing a venipuncture site, unsightly blemishes, and other undesired temporary and permanent outcomes, including infection or eventual loss of use entirely. Eventually, this damage may result in the vein collapsing. Even with these risks, many chronic illnesses must be treated with regular venipunctures, resulting in individuals who may end up suffering from, e.g., progressively scarred veins and vein collapse. Additionally, certain medical procedures, such as dialysis fistula creation, rely on strong, healthy veins and may require venipuncture in specific or known locations. Patients with progressively scarred veins may have difficulty or be completely unable to receive the treatment they need for chronic illness, due to vascular damage over time.

An individual may receive venipunctures from a variety of individuals (i.e., doctors, nurses, themselves, or any other individual) and in a variety of settings (i.e., hospitals, labs, doctor's offices, home, or other locations.) By receiving venipunctures in this manner, no centralized manner of tracking an individual's venipunctures exists. The recollection of individuals involved in venipuncture is limited in many ways. By not tracking venipuncture locations over time, a medical professional or the patient cannot be informed when it may be safe to return to a previously used site or how far away from an earlier venipuncture a subsequent use is advisable.

Accordingly, there is a need to provide technological solutions addressing the specific risks associated with repeated venipunctures.

### SUMMARY

The disclosed subject matter may calculate and display a venipuncture risk zone on a map of a user's body based upon historical venipuncture data associated with that user. In an example, an apparatus may include a processor, and a memory coupled with the processor that effectuates operations. The operations may include capturing an image of a location on user's body (e.g., back of hand, arm, leg, etc.), retrieving historical venipuncture data (e.g., puncture site, needle gauge, time of injection, treatment impact, etc.), calculating based on the data, one or more local effects on the location on the user's body, calculating based on the venipuncture data and one or more local effects, one or more composite effects on at least the location on the user's body, calculating a risk zone on at least the location on the user's body, calculating a risk zone decay, and displaying the risk zone superimposed on the image of a location on the body.

Additional advantages will be set forth in part in the description which follows or may be learned by practice. The advantages will be realized and attained by means of the elements and combinations particularly pointed out in the appended claims. It is to be understood that both the foregoing general description and the following detailed description are example and explanatory only and are not restrictive or in any way limiting.

This Summary is intended to describe only certain aspects of the disclosure and should not be interpreted as in any way limiting the disclosure or scope or spirit of the invention.

### BRIEF DESCRIPTION OF THE DRAWINGS

A more detailed understanding may be had from the following description, given by way of example in conjunction with the accompany drawings wherein:

FIG. 1 illustrates an example system that may enable systems for tracking venipunctures, calculating venipuncture risk, and displaying said risk for the user.

FIG. 2 illustrates an example method that may enable systems for tracking venipunctures, calculating venipuncture risk, and displaying said risk for the user.

FIG. 3 illustrates an example method that may enable systems for determining suitable sites for injection of a particular treatment, calculating the local and composite effects from the injection at each suitable injection site, and selecting a recommended suitable injection site for the user.

FIG. 4 illustrates an example system that may enable systems for tracking venipunctures, calculating venipuncture risk, and displaying said risk for the user.

The details above in the Brief Description of the Drawings are intended to describe only some aspects relating to certain embodiments of the innovations herein and should not be deemed in any way limiting with respect to requiring or omitting any aspect for embodiments to be claimed or otherwise limiting the disclosure or embodiments keeping with its scope or spirit.

### DETAILED DESCRIPTION

Vein punctures (venipunctures) are a necessary part of modern healthcare. From routine blood draws to treating various chronic illnesses, individuals will undergo numerous venipunctures throughout their lives. Each venipuncture, however, can result in permanent damage and scarring where the needle pierces the vascular wall. Over time, if an individual continues to receive venipunctures in the same location, scarring and damage may lead to conditions or persistent injuries, including vein collapse. Vein collapse can critically undermine treatment for many chronic illnesses, which rely on healthy veins to support long-term access for blood draws or for intravenous injection.

To mitigate vein collapse and lasting damage, methods to track venipuncture sites are required. However, as individuals may receive venipunctures from various individuals (e.g., nurses, doctors, self-administered, etc.) and in various locations (e.g., clinics, hospitals, doctor's offices, at home, etc.), it would be advantageous for such a tracking system to follow the individual. To best serve the individual, it is often helpful to permit them to self-manage portions of their care or records. In this regard, the tracking system may be designed to avoid the use of complicated medical devices and systems, allowing it to be managed by a person with limited medical knowledge and be affordable to the general public. In embodiments, though, more specialized devices can be used, and data or records related thereto can be provided to the patient and ingested into a solution as disclosed herein. The disclosed subject matter provides methods for calculating the risk of injection in a specific location on the body based on a history of past injections, and displays this risk on an image of that body location without requiring complicated medical imaging or expensive, highly specialized devices.

Additionally, some organizations and communities have begun seeking harm reduction programs and tools to mitigate health consequences tied to drug use, including intravenous drug injection. One harm of intravenous drug use is lasting vein damage due to repeated venipunctures. Providing these individuals a way to track their injections may encourage them to rotate injection sites thus reducing health risks and improving the ability of medical professionals to provide them proper care.

As used in this application, “venipuncture” and “injection” have coterminous definitions and uses. Both words are used to describe a needle, or a similar sharp object, piercing the wall of a vein.

The disclosed subject matter may enable the use of a user input device (e.g., phone, tablet, photo or video camera, computer, smart wearable, or similar devices) to track a user's injection data and display a risk profile for various possible injection sites on the user's body.

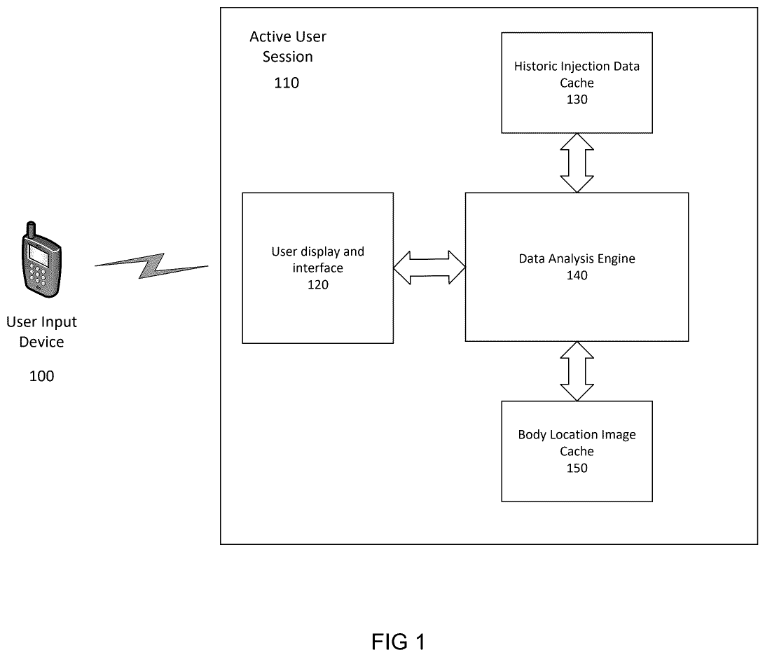

FIG. 1 illustrates an example system for tracking venipunctures as disclosed herein. System 100 includes a user input device 110 coupled with active user session 120. User input device 110 may be communicatively coupled with active user session 120 via wi-fi, Bluetooth, cellular, near-field communication, or similar means. Active user session 120 may also be, in whole or in part, programmatically stored on user input device 110 and interface with an end user as any program on user input device 110. Active user session 120 may contain one or several of user display and interface 130, historic injection data cache 140, data analysis engine 150, and body location image cache 160.

In some embodiments user display and interface 130 may be communicatively coupled with data analysis engine 150 via wi-fi, Bluetooth, cellular, near-field communication, or similar means.

In alternative embodiments, user display and interface 150 and data analysis engine 150 may both be programmatically stored on user input device 110. Data analysis engine 150 may then be communicatively coupled with one or both of historic injection data cache 140 and body location image cache 160 via wi-fi, Bluetooth, cellular, near-field communication, or similar means. Such embodiments and systems are set forth for example purposes only, and those of skill in the art will on review of the disclosures herein appreciate that various devices, sensors, communication techniques, platforms, et cetera, can be used to implement the solutions herein in various combinations other than those described without departing from the scope or spirit of the innovation.

User input device 110 may send, receive, or store venipuncture information to and from active user session 120. The stored and communicated information may include injection data, historical injection data, body location, body location images, treatment impact information, risk zones, risk zone decay or other similar information and data.

Injection data may include data associated with an injection such as, injection location data mapped to a body receiving the injection, a time of injection for the injection, and treatment impact data for the injection. Treatment impact data may include the needle gauge used for the injection, substance(s) being injected and volume of said substance(s), vascular dimensions of patient, health complications that may impact vascular health, or similar factors that may increase or decrease the damage an injection may cause on the user's vascular health. The substance(s) being injected and volume of said substances(s) may have more or less impact, cause more or less inflammation of the injection site, cause more or less damage to vascular health, increase or decrease healing time after the injection, amongst other impacts. Historical injection data may include the injection data for a user from one or more previous injections.

Body location may include a point in space on a 3d model of a body part, a coordinate on a coordinate system imposed on a 2d picture or 3d model of a body part, a distance measured from identifiable points (e.g., bone protrusions, scars, birthmarks, edges of body part, other body parts, amongst other identifying features). The body location may be captured by a user lining up a body part image in a template or outline containing coordinates; entering measurements that the user manually measured; using imaging sensors with directions on how to align, hold, or move the camera or LIDAR sensor to capture a specific orientation or position of the body part; applying stickers or similar marks on the body part to identify specific positions; placing the body part near a scaling object to establish size. The captured body location may be used with past or future captured body locations to build a body image model. The body image model may adapt over time to detect natural changes in a user's body and migrate relative locations or modify appearance based on changes to the body, age, or similar events. In some embodiments, once a body location is captured for the first time, future body location data may include just the data for a location on an already captured or generated body location model or image.

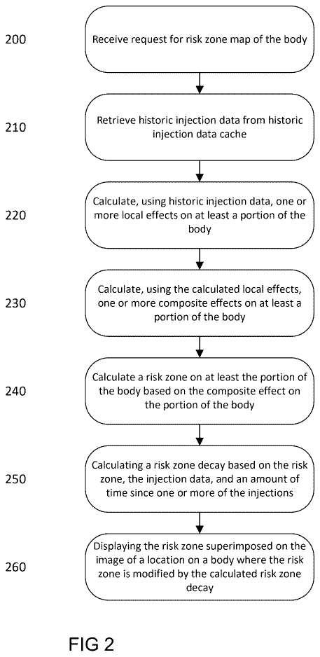

FIG. 2 illustrates an example method that may enable a system to track a user's injection data and display a risk profile for various possible injection sites on the user's body. At step 200 active user session 120 may receive a request to display vascular health risk zones with a risk zone map. The request to display vascular health risk zones may be received from either user input device 110 or user display and interface 130. The request may be for the health risk zones over a portion of a body, over key areas of the body, over the entire body, or any combination thereof.

At step 210, in response to the request to display vascular health risk zones, data analysis engine 150 may retrieve historic injection data stored in historic injection data cache 140. The historic injection data retrieved may be only the historic injection data associated with the portion of a body for which the request to display vascular health risk zones was made. The historic injection data stored in historic injection data cache 150 may include injection data related to one or more previous injections. The injection data related to one or more previous injections may be added to the historic injection data cache one previous injection at a time or all previous injections at the same time, in one data transfer. Data for individual previous injections may be appended to the historic injection data cache at the time of injection by receiving the data from the user input device 110 or user display and interface 130.

At step 220, data analysis engine 150 may calculate, using said historic injection data, one or more local effects on at least a portion of the body. The local effects on at least a portion of the body may be calculated based upon treatment impact data for one or more injections. After calculating the one or more local effects on at least a portion of the body, at step 230 data analysis engine may use the calculated local effects to calculate one or more composite effects on at least a portion of the body. Data analysis engine 150 may calculate said composite effects by using one or more local effects, the locations of the one or more local effects, the proximity of the one or more local effects, the treatment impact of the injections making up the one or more local effects, and similar data.

At step 240, data analysis engine 150 may calculate a risk zone on at least a portion of the body based upon the composite effect on the portion of the body calculated in step 230. The risk zone is defined as an area or areas near historic injection locations (as logged by the historic injection data) where a subsequent injection may risk undesired vascular scarring or other damage.

In some embodiments, the risk zone calculated during step 240 may be based upon a user's particular health diagnosis. Data analysis engine 150 may assess a likelihood that a user will need subsequent injections over a long period of time and create a risk zone to preserve certain portions of the body for later venipuncture. Such likelihood can be based on input from the user, a medical provider, a data source, et cetera, that treatment is ongoing (e.g., indefinite or part of a course of treatments); the nature of the treatment (e.g., treatment that is typically administered in a series); the injection data; or other bases. In embodiments, future injections are known to be definite. Data analysis engine 150 may also consider the likelihood that the user may need future health interventions that rely upon healthy veins in particular areas such as chemotherapy, dialysis, medically inserted fistulas, frequent intravenous infusions, or similar treatments.

In another example, the risk zone calculated by data analysis engine 150 may be customizable to decrease risk shortly after an injection but then increasing the risk zone after a set period of time. This may encourage users to use nearby veins in one location of the body before rotating to another for later injections.

At step 250, data analysis engine 150 may calculate a risk zone decay based on the risk zone, the injection data, and an amount of time since one or more of the injections. Data analysis engine 150 may increase the risk zone decay (e.g., longer half-life) in relation to the time since a previous injection. Comparatively, data analysis engine 150 may decrease the risk zone decay (e.g., shorter half-life) in relation to the recency of an injection. The treatment impact, associated with the historic injection data, may also be used by data analysis engine 150 to calculate the risk zone decay. This may modulate the risk zone based on the causticity of the chemical used during the injection, the needle gauge, the time length the injection was inserted, or similar treatment impact data. For example, data analysis engine 150 may increase the risk zone decay based on injection data indicating a non-caustic chemical was used in the previous injection and a set period of time has passed since the last injection. In another example, data analysis engine 150 may decrease the risk zone decay based on injection data indicating a caustic chemical was used in the previous injection and a set period of time has not passed since the last injection. The models and data referenced and used herein may inform the qualitative aspects of each individual patient or treatment instance.

In some embodiments the risk zone and risk zone decay may be calculated by reference to tabulated treatment impact data. The tabulated data may be absolute based upon the treatment impact data or arranged by demographic segment (e.g., age, gender, height, weight, race, and other demographic identifiers). The tabulated data may provide impact indices which calculate a relative treatment impact of various treatments at various dosages and frequencies. Data analysis engine 150 may sum or otherwise calculate with these indices to determine the local and composite effects of the treatment impact data received by data analysis engine 150. For example, an impact index calculated for a non-caustic injected chemical when it is injected at low frequency may lead data analysis engine to decrease the calculated risk zone or increase the calculated risk zone decay. In another example, the treatment impact indices for a combination of two injected chemicals may lead data analysis engine to increase the calculated risk zone or decrease the calculated risk zone decay due to the potential for a reaction between the injected chemicals.

Data analysis engine 150 may also use the tabulated treatment impact data to calculate local or composite impacts of various treatment impact data. In alternative or complimentary embodiments, there may be a model of the local or composite effects. The model may provide outputs for the effects or harm of a venipuncture over time. These outputs may be a snapshot of the impact of a venipuncture at different times (e.g., immediately thereafter, 48 hours later, weeks later, or other time periods) or a continuous period of time that covers the injection through substantial healing. The model may be trained on venipuncture data reflective of venipuncture impact on individuals with various injuries, conditions, or treatment (e.g., anemia, chemotherapy, immunodeficiency, crush syndrome, or other similar medical complications.) The model may recalculate local or composite effects based upon the outcomes of prior venipunctures on similar, or different, individuals.

In alternative or complimentary embodiments, a user can feed their own data into a treatment impact model including images; sensor data; pain experienced (at the time of the injection and in the time after the injection); inflammation of the injection site; closure of the injection site; seepage, pus, or other sign of infection of the injection site. This user data may be used to create an impact model reflective of the treatment impact of venipunctures on a specific user. Data analysis engine may use the user impact model to calculate the risk zone, the risk zone decay, or other impact models.

At step 260, user display and interface 130 may receive from data analysis engine 150 a calculated local effect, composite effect, risk zone, and risk zone decay. User display and interface 130 may use the received data to display the risk zone or injection data. The risk zone or injection data may be superimposed on the image of a location on a body to show the user where a subsequent venipuncture may result in undue harm. In one example, user display and interface 130 may display the risk zone or injection data through the use of an augmented reality display or projection, or an overlay on a stored picture of the user's body part or a representation thereof. After displaying the risk zone or injection data for the user, user display and interface 130 may receive from the user device 110 new injection data which may be appended to historic injection data cache 140.

In another example, user display and interface 130 additionally may display recommended injection locations superimposed on the image of a location on a body based upon the calculated risk zone, risk zone decay, and historic injection data. Said recommended injection locations may also be based upon a user's likelihood of needing certain medical treatments in the future due to their diagnosis, risk factors, or other similar factors.

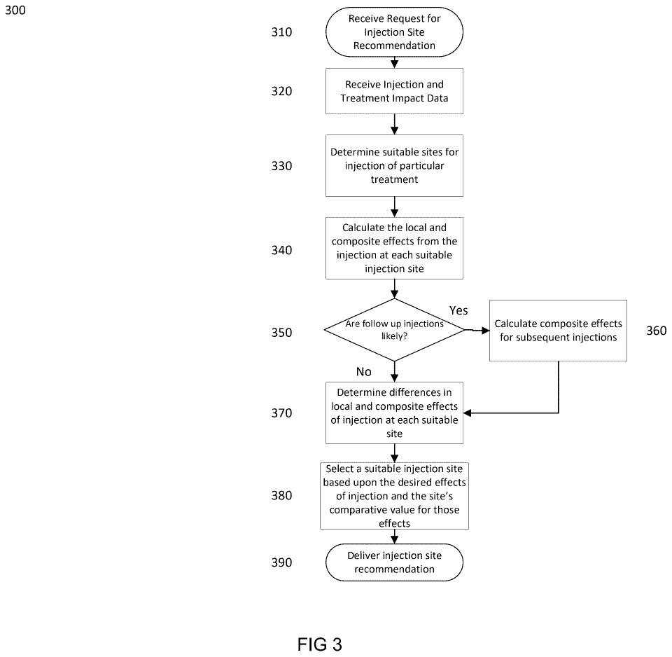

FIG. 3. illustrates an example method 300 that may enable a system to calculate a suitable injection site based upon injection and treatment impact data and display an injection site recommendation. Method 300 may be undertaken by data analysis engine 150, which may function locally, be a distributed system, or be fully remote. At step 310, the user, active user session 120, or data analysis engine 150, or other input method may request an injection site recommendation. At step 320, the user, active user session 120, data analysis engine 150, or other input method may provide method 300 the injection and treatment impact data for an imminent venipuncture. At step 330, method 300 may determine a set of potential suitable sites for the venipuncture based upon the injection data, treatment impact data. Method 300 may determine potential suitable sites based upon the treatment type. For example, method 300 may determine potential suitable sites are located on the back of a user's hand because the treatment is a blood draw. In another example, method 300 may determine potential suitable sites exist in the user's cubital fossa as the treatment is a blood donation which requires higher volume extracted through the venipuncture.

At step 340, method 300 may calculate the local and composite effects from the imminent injection at each suitable injection site. Method 300 may use any of the means or methods referenced herein in calculating the local or composite effects of the imminent injection through the use of the treatment and treatment impact data. In some embodiments, at decision 350 method 300 may determine if any follow-up injections are likely. Method 300 may determine this based upon user data, treatment data, treatment impact data, or similar. For example, if the treatment data indicates the user is receiving a venipuncture for chemotherapy, method 300 may determine that follow-up injections are likely. In another example, the user data may indicate the user has a chronic illness requiring many blood tests and therefore follow-up injections are likely. In another example, the treatment impact data may indicate the chemical injected has significant impact where the user should not receive further injections for a period of time after the venipuncture. If method 300 determines that follow-up injections are likely, it may proceed to step 360 where it may calculate the composite effects for subsequent injections before proceeding to step 370. If method 300 determines follow-up injections are not likely, it may immediately proceed to step 370.

At step 370, method 300 may determine differences in the local and composite effects of the injection at each potential suitable site. For example, method 300 may determine that a venipuncture will result in more vascular damage to the back of a user's hand than in the user's cubital fossa due to the amount of blood to be extracted by a blood draw. In another example, method 300 may determine that the composite effect will increase in one of the potential suitable sites due to the causticity of the chemical injected and the potential suitable site's proximity to a vital organ.

At step 380, method 300 may select a recommended suitable injection site from the set of potential suitable injection sites. In making this selection, method 300 may consider the desired effects of the injection based upon the injection and treatment impact data as well as by considering the suitable injection site's comparative value for those effects. For example, method 300 may select a recommended injection site on the back of the user's hand as the treatment data indicates the injection will likely be self-administered and this site is easier for a non-medically trained user to access. In another example, method 300 may select a recommended injection site as the treatment data indicates the injection is preferred when the user is in a particular position (e.g., prone, sitting down, standing up) and the recommended injection site is preferred for that particular position.

At step 390, method 300 returns a recommended injection site to active user session 120, data analysis engine 150, or any alternative external or internal processor which requested the recommended injection site at step 310.

In some embodiments, user display and interface 130 may recommend a next shot location based upon the next injection's treatment impact. Alongside other factors such as the calculated risk zone and risk zone decay, as well as the historic injection data, the user's medical treatment information and diagnoses, and other similar data.

In further embodiments, user display and interface 130 may display guidance for the user on administering a next injection. The guidance for the user may be based upon received next injection treatment impact or other data such as a user's health diagnosis or likelihood of requiring future injections.

In further embodiments, data analysis engine 150 may build an individualized user model based upon user input data over a period of time. The user input data may include the user's response to each injection based upon the substance injected, gauge of the needle used for the injection, time duration of the injection, body location of the injection, the time it took the user's body to recover from the injection, or similar injection-related data. Data analysis engine 150 may build the individualized user model through data provided by active user session 120 for individualized injection events or data analysis engine 150 may build the individualized user model based upon a training set of user input data. Data analysis engine 150 may build the individualized user model based upon user input data focused on how the user reacts to a specific medicine, a specific treatment, or how the user reacts to venipunctures and injections generally.

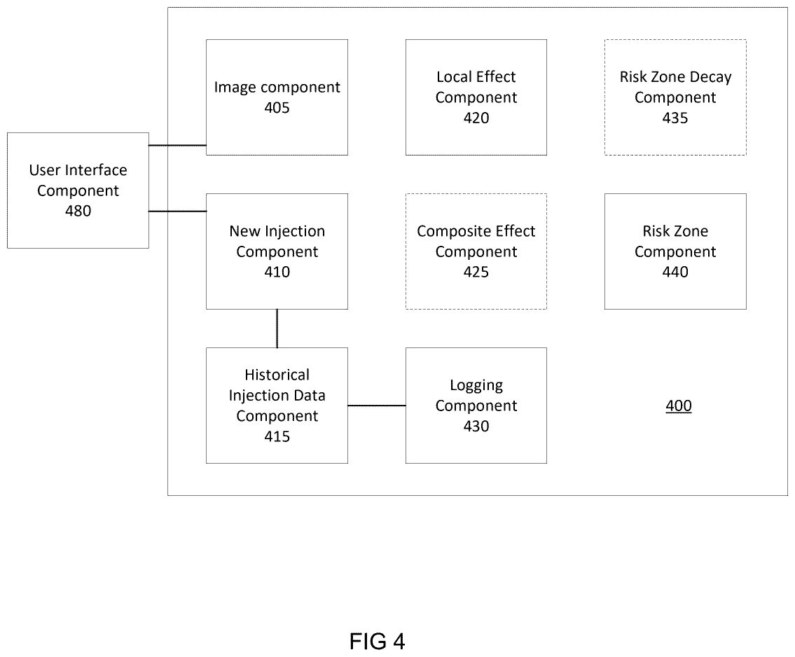

FIG. 4 illustrates a system 400 disclosed herein. System 400 comprises user interface component 480, image component 405, new injection component 410, historical injection data component 415, local effect component 420, composite effect component 425, logging component 430, risking zone decay component 435, and risk zone component 440.

User interface component 480 is configured to facilitate user input to system 400 and display output from system 400 to a user. User interface component 480 may also be configured to display a risk zone calculated by risk zone component 440 and risk zone decay component 435. Image component 405 is configured to capture an image of a location on a body. Historical injection data component 415 is configured to retrieve historical injection data for one or more injections for said location captured by image component 405. Historical injection data may include location data mapped to a body receiving the one or more injections, a needle gauge for each of the one or more injections, a time of injection for each of the one or more injections, and treatment impact for each of the one or more injections.

Local effect component 420 may be configured to calculate, for each of the one or more injections and based on the injection data, one or more local effects on at least a portion of the body. Composite effect component 425 may be configured to calculate, based on the injection data and the local effects, one or more composite effects on at least a portion of the body.

Risk zone component 440 may be configured to calculate a risk zone on at least the portion of the body based on the composite effect calculated by composite effect component 425. Risk zone decay component 435 may be configured to calculate a risk zone on at least a portion of the body based on the risk zone calculated by risk zone component 440, the injection data, and an amount of time since one or more of the injections tracked by historical injection data component 415.

New injection component 410 may be configured to receive new injection data and provide logging component 430 the new injection data to be appended to the historical injection data for a location on the body. The new injection data may include location data mapped to a body receiving one or more injections, a needle gauge for each of the one or more injections, a time of injection for each of the one or more injections, and treatment impact for each of the one or more injections.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present disclosure, example methods and materials are now described.

It must be noted that as used herein and in the appended claims, the singular forms “a,” “an,” and “the” include plural referents unless the context clearly dictates otherwise. Thus, for example, reference to “a stimulus” includes a plurality of such stimuli and reference to “the signal” includes reference to one or more signals and equivalents thereof as known to those skilled in the art, and so forth. In addition, the use of the word “or” is generally used inclusively unless otherwise provided herein.

In describing preferred examples of the subject matter of the present disclosure, as illustrated in the Figures, specific terminology is employed for the sake of clarity. The claimed subject matter, however, is not intended to be limited to the specific terminology so selected, and it is to be understood that each specific element includes all technical equivalents that operate in a similar manner to accomplish a similar purpose.

This written description uses examples to disclose the invention, including the best mode, and to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

One skilled in the art will appreciate further features and advantages based on the described examples. Accordingly, the disclosure is not to be limited by what has been particularly shown and described, except as indicated by the appended claims. Further, although at least one series of steps are presented as an example method of practicing one or more examples described herein, it will be appreciated by those skilled in the art that the steps identified may be practiced in any order that is practicable, including without limitation the omission of one or more steps.

What is claimed is:

1. An apparatus, comprising:
a processor; and
memory coupled with the processor, the memory storing executable instructions that when executed by the processor cause the processor to effectuate operations comprising:
capturing an image of a location on a body;
retrieving historical injection data for said location on said body comprising, for one or more injections, location data mapped to a body receiving the one or more injections, a needle gauge for each of the one or more injections, a time of injection for each of the one or more injections, and treatment impact for each of the one or more injections;
calculating, for each of the one or more injections and based on the injection data, one or more local effects on at least a portion of the body;
calculating, based on the injection data and the local effects, one or more composite effects on at least the portion of the body;
calculating a risk zone on at least the portion of the body based on the composite effect on the body;
calculating a risk zone decay based on the risk zone, the injection data, and an amount of time since one or more of the injections;
displaying the risk zone superimposed on the image of a location on a body, wherein display of the risk zone is based on the zone decay;
receiving new injection data comprising, for one receiving injection data comprising, for one or more injections, location data mapped to a body receiving the one or more injections, a needle gauge for each of the one or more injections, a time of injection for each of the one or more injections, and treatment impact for each of the one or more injections; and
appending said new injection data to the historical injection data for said location on said body.

2. The apparatus of claim 1, wherein the image of a location on a body displays ideal injection sites based on the calculated risk zones, risk factors, and injection site locations.

3. The method of claim 2, wherein the image of a location on a body displays ideal shot sites based on the patient's likelihood of needing any number of future injections.

4. The method of claim 2, comprising:
receiving next shot data;
based on the next shot data and patient vasculature, identifying a recommended injection site; and
displaying the recommended injection site on the map of at least the portion of the body.

5. The method of claim 4, comprising:
displaying guidance for administering a next shot at the recommended injection site.

6. The method of claim 5, wherein the guidance comprises a real time augmented reality view displaying placement for the next shot.

7. The method of claim 1, wherein the calculated risk zones are a gradient from lowest to highest risk.

8. A method comprising:
receiving injection data comprising, for one or more injections, location data mapped to a body receiving the one or more injections, a needle gauge for each of the one or more injections, a time of injection for each of the one or more injections, and treatment impact for each of the one or more injections;
calculating, for each of the one or more injections and based on the injection data, one or more local effects on at least a portion of the body;
calculating, based on the injection data and the local effects, one or more composite effects on at least the portion of the body;
calculating a risk zone on at least the portion of the body based on the composite effect on the body;
calculating a risk zone decay based on the risk zone, the injection data, and an amount of time since one or more of the injections; and
displaying the risk zone superimposed on a map of at least the portion of the body, wherein display of the risk zone is based on the zone decay.

9. The method of claim 8, wherein the image of a location on a body displays ideal shot sites based on the calculated risk zones, risk factors, and injection site locations.

10. The method of claim 8, wherein the image of a location on a body displays ideal shot sites based on the patient's likelihood of needing any number of future injections.

11. The method of claim 8, comprising:
receiving next shot data;
based on the next shot data and patient vasculature, identifying a recommended injection site; and
displaying the recommended injection site on the map of at least the portion of the body.

12. The method of claim 11, comprising:
displaying guidance for administering a next shot at the recommended injection site.

13. The method of claim 12, wherein the guidance comprises a real time augmented reality view displaying placement for the next shot.

14. The method of claim 8, wherein the calculated risk zones are a gradient from lowest to highest risk.

15. A system, comprising:
A user interface component configured to facilitate user input to the system and display system output for a user
an image component configured to capture an image of a location on a body;
a historical injection data component configured to retrieve historical injection data for said location on said body comprising, for one or more injections, location data mapped to a body receiving the one or more injections, a needle gauge for each of the one or more injections, a time of injection for each of the one or more injections, and treatment impact for each of the one or more injections;
a local effect component configured to calculate, for each of the one or more injections and based on the injection data, one or more local effects on at least a portion of the body;
a composite effect component configured to calculate, based on the injection data and the local effects, one or more composite effects on at least the portion of the body;
a risk zone component configured to calculate a risk zone on at least the portion of the body based on the composite effect on the body;
a risk zone decay component configured to calculate a risk zone decay based on the risk zone, the injection data, and an amount of time since one or more of the injections;
a user interface component configured to display the risk zone superimposed on the image of a location on a body, wherein display of the risk zone is based on the zone decay;
a new injection component configured to receive new injection data comprising, for one receiving injection data comprising, for one or more injections, location data mapped to a body receiving the one or more injections, a needle gauge for each of the one or more injections, a time of injection for each of the one or more injections, and treatment impact for each of the one or more injections; and
a logging component configured to append new injection data to the historical injection data for said location on said body.

16. The system of claim 15, wherein the user interface component displays, on an image of a location on a body, ideal shot sites based on the calculated risk zones, risk factors, and injection site locations.

17. The system of claim 15, wherein the user interface component displays, on an image of a location on a body, ideal shot sites based on the patient's likelihood of needing any number of future injections.

18. The system of claim 15, comprising:
The new injection component receiving next shot data;
based on the next shot data and patient vasculature, the risk zone component identifying a recommended injection site; and
the user interface component displaying the recommended injection site on the map of at least the portion of the body.

19. The system of claim 18, comprising:
The user interface component displaying guidance for administering a next shot at the recommended injection site.

20. The system of claim 19, wherein the guidance comprises a real time augmented reality view displaying placement for the next shot.
